(19) United States
(12) Patent Application Publication
(10) Pub. No.: US 2021/0110307 A1
(43) Pub. Date: Apr. 15, 2021

(54) ELECTRON DENSITY ESTIMATION METHOD, ELECTRON DENSITY ESTIMATION APPARATUS, AND RECORDING MEDIUM

(71) Applicant: Panasonic Intellectual Property Management Co., Ltd., Osaka (JP)

(72) Inventors: KENSUKE WAKASUGI, Tokyo (JP); KOJI MORIKAWA, Tokyo (JP)

(21) Appl. No.: 17/128,298

(22) Filed: Dec. 21, 2020

Related U.S. Application Data
(63) Continuation of application No. PCT/JP2019/032350, filed on Aug. 20, 2019.

(30) Foreign Application Priority Data
Sep. 3, 2018 (JP) 2018-164752

(51) Int. Cl. G06N 20/00

(52) U.S. Cl. CPC G06N 20/00

(57) ABSTRACT

An electron density estimation method includes calculating a first electron density by inputting first input data to an electron density estimator, performing a numerical simulation using the first input data and the first electron density to calculate a second electron density, the numerical simulation being processing in which the first input data and the first electron density are set as initial values and in which electron density update processing using a density functional method is performed one or more times, the second electron density being not a convergence value obtained by the density functional method, performing learning for the estimator by calculating a parameter for the estimator, the parameter minimizing a difference between the first and second electron densities, obtaining, from a database, and inputting second input data to the estimator, in which the parameter is set, to estimate a third electron density, and outputting the third electron density.

### BACKGROUND

### 1. Technical Field

The present disclosure relates to a technology for estimating the electron density of a material.

### 2. Description of the Related Art

Hitherto, a technology called a density functional method has been developed in which, on the basis of physical laws, the calculation for updating the density of electrons in a material is repeated, and a convergence value for the density of electrons is obtained (for example, see “Kohn, W., & Sham, L. J. (1965). Self-consistent equations including exchange and correlation effects. Physical review, 140 (4A), A1133” (hereinafter referred to as NPL 1)). In addition, a technology for developing a catalyst using a density functional method is also known (for example, see Japanese Patent No. 6015336). In this manner, density functional methods are used as tools for developing new materials.

In recent years, a technology for estimating an electron density using an estimator obtained by machine learning has been developed. For example, a method for estimating an electron density from the potential in a material is disclosed (see “Brockherde, F., Vogt, L., Li, L., Tuckerman, M. E., Burke, K., & Mueller, K. R. (2017). Bypassing the Kohn-Sham equations with machine learning. Nature communications, 8 (1), 872” (hereinafter referred to as NPL 2)). Specifically, in NPL 2, a technology is disclosed in which about a specific material a convergence value for the electron density is calculated by changing the distance and angle between atoms in a density functional method, an estimator that estimates an electron density is trained using the calculated convergence value for the electron density, and the electron density for every distance and angle between atoms is estimated using the obtained estimator.

A method for estimating the energy of electrons from the potential in a material is disclosed (see “Mills, K., Spanner, M., & Tamblyn, I. (2017). Deep learning and the Schroedinger equation. Physical Review A, 96 (4), 042113” (hereinafter referred to as NPL 3)). Specifically, in NPL 3, a technology is disclosed in which the energy of electrons obtained from a two-dimensional random potential or a potential well by solving the Schrödinger equation is estimated using a deep neural network.

### SUMMARY

However, the existing technologies described above require an electron density convergence value as training data for an estimator and thus need to be further improved.

One non-limiting and exemplary embodiment provides a technology for training an electron density estimator without using an electron density convergence value.

In one general aspect, the techniques disclosed here feature an electron density estimation method according to an aspect of the present disclosure is an electron density estimation method for an electron density estimation apparatus that estimates an electron density of a material from material information regarding a composition and a structure of the material, the electron density estimation method causing a computer of the electron density estimation apparatus to execute a process, the process including: (a1) obtaining first input data from a training database in which the material information is stored, (a2) calculating a first electron density by inputting the first input data to an electron density estimator, (a3) performing a numerical simulation using the first input data and the first electron density to calculate a second electron density, the numerical simulation being processing in which the first input data and the first electron density are set as initial values and in which electron density update processing using a density functional method is performed one or more times, the second electron density being not a convergence value obtained by the density functional method, (a4) performing learning for the electron density estimator by calculating a parameter for the electron density estimator, the parameter minimizing a difference between the first electron density and the second electron density; (a5) obtaining second input data from a test database in which the material information is stored, and inputting the second input data to the electron density estimator, in which the parameter is set, to estimate a third electron density, and (a6) outputting the estimated third electron density.

According to the present disclosure, the electron density estimator can be trained such that an output value from the electron density estimator approaches an electron density convergence value obtained by a density functional method without using the electron density convergence value obtained by the density functional method.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating an example of specific electron density values;

FIG. 2 is a diagram schematically illustrating the way in which the density of electrons is updated by repeating an electron density update calculation in a density functional method;

FIG. 3 is a diagram illustrating a difference calculated when an electron density estimator is trained;

FIG. 4 is a diagram schematically illustrating the way in which an estimation value obtained by an electron density estimator is set as an initial electron density in a density functional method and the density of electrons is updated by repeating an electron density update calculation;

FIG. 5 is a schematic diagram illustrating the training of an electron density estimator using an update value as teacher data, the update value being obtained by performing an electron density update calculation one time in a density functional method using an estimation value;

FIG. 6 is a diagram illustrating the progress of learning in a case where the number of pieces of training data is changed for a classification problem using a natural image data set called the CIFAR-10 data set;

FIG. 7 is a block diagram illustrating the configuration of an electron density estimation system according to an embodiment of the present disclosure;

FIG. 8 is a block diagram illustrating a detailed configuration of an electron density updater in FIG. 7;

FIG. 9 is a diagram illustrating an example of first input data described in a format called CIF;

FIG. 10 is a diagram illustrating an example of the first input data described in a format called POSCAR;

FIG. 11 is a diagram illustrating an example of a descriptor;

FIG. 12 is a diagram illustrating an example of an initial electron density, a first electron density, and the electron density estimator;

FIG. 13 is a diagram illustrating an example of processing in which a second electron density is calculated from the first electron density;

FIG. 14 is a diagram illustrating an example of processing in which a third electron density is estimated;

FIG. 15 is a flow chart illustrating the overall procedure of processing performed by an electron density estimation apparatus in a first embodiment of the present disclosure;

FIG. 16 is a flow chart for describing details of processing performed by the electron density updater in step S102 of FIG. 15;

FIG. 17 is a diagram for describing processing in a second embodiment of the present disclosure;

FIG. 18 is a diagram for describing processing in the second embodiment of the present disclosure;

FIG. 19 is a flow chart illustrating an example of processing according to the second embodiment of the present disclosure;

FIG. 20 is a diagram illustrating the unit cell vectors, the atomic coordinates, the atomic number, the atomic radius, and the like of Al₂O₃; and

FIG. 21 is a diagram illustrating, for example, an example of a descriptor of an Al crystal expressed by a combination of first input data and the atomic number or the atomic radius.

### DETAILED DESCRIPTION

### Underlying Knowledge Forming Basis of the Present Disclosure

When a new material is developed, it is necessary to estimate characteristic values of the material such as a band gap and formation energy. In order to do that, it is necessary to calculate the density of electrons in the material, and thus the density functional method described in NPL 1 has been previously used as an effective tool for developing new materials (for example, see Japanese Patent No. 6015336).

In contrast, a method has been recently proposed in which the density and energy of electrons are estimated from the potential of a material by using machine learning techniques such as a deep neural network, kernel ridge regression, and a support vector machine. In this method, an electron density convergence value obtained by a density functional method or an electron energy convergence value is prepared, and an estimator is trained by updating a parameter for the estimator such that the error between the convergence value and an estimation value is minimized.

For example, in NPL 2, a technology is disclosed in which, about a material having a specific structure, an electron density estimator is trained using an electron density convergence value calculated by a density functional method.

However, in NPL 2, the estimator is trained only about, for example, a material having a specific structure such as a perovskite structure, and thus there is a problem in that the density of electrons cannot be accurately estimated for an unknown material having a different structure from a learned material.

In addition, it is said that with the current computing power it takes about three weeks to obtain a convergence value for just one material by using a density functional method, which means it takes about a few years to obtain convergence values of many materials needed for training. Thus, the method described in NPL 2 for obtaining a convergence value by the density functional method has a problem in that it takes massive time to collect teacher data.

In NPL 3, a technology is disclosed in which the energy of electrons obtained by solving the Schrödinger equation is estimated using a deep neural network.

However, in NPL 3, an enormous volume of training data is needed to train a deep neural network, and thus there is a problem in that it takes massive time to prepare training data. In addition, NPL 3 does not disclose that a deep neural network is trained without using a convergence value obtained by a density functional method.

Note that NPL 1 is a prior art document disclosing basic techniques about density functional methods and does not disclose that an estimator is trained through machine learning. In addition, Japanese Patent No. 6015336 only discloses that a density functional method indicated in NPL 1 is used when comparative binding energy of anchor particles and noble metal particles at 900° C. is calculated, and does not disclose that an estimator is trained through machine learning. Furthermore, the technologies described below are disclosed in prior art documents (see “Finger, L. W., & Hazen, R. M. (1978). Crystal structure and compression of ruby to 46 kbar. Journal of Applied Physics, 49 (12), 5823-5826” (hereinafter referred to as NPL 4) and “Cicek, oe., Abdulkadir, A., Lienkamp, S. S., Brox, T., & Ronneberger, O. (2016, October). 3D U-Net: learning dense volumetric segmentation from sparse annotation. In International Conference on Medical Image Computing and Computer-Assisted Intervention (pp. 424-432). Springer, Cham.” (hereinafter referred to as NPL 5)).

The inventors considered a calculation for obtaining an electron density update value in a density functional method and the update of a parameter for an electron density estimator through machine learning, and found that, without using an electron density convergence value obtained by a density functional method, an electron density estimator can be trained such that an output value from the electron density estimator approaches the electron density convergence value obtained by the density functional method, by implementing calculation processing for obtaining an electron density update value into processing for training the electron density estimator. As a result, the inventors have conceived the present disclosure.

That is, an object of the present disclosure is to provide a technology that makes it possible to train an electron density estimator such that an output value from the electron density estimator approaches an electron convergence value obtained using a density functional method without using the electron density convergence value obtained by the density functional method.

An electron density estimation method according to an aspect of the present disclosure is an electron density estimation method for an electron density estimation apparatus that estimates an electron density of a material from material information regarding a composition and a structure of the material,

the electron density estimation method causing a computer of the electron density estimation apparatus to execute a process, the process including:

(a1) obtaining first input data from a training database in which the material information is stored,

(a2) calculating a first electron density by inputting the first input data to an electron density estimator,

(a3) performing a numerical simulation using the first input data and the first electron density to calculate a second electron density, the numerical simulation being processing in which the first input data and the first electron density are set as initial values and in which electron density update processing using a density functional method is performed one or more times, the second electron density being not a convergence value obtained by the density functional method,

(a4) performing learning for the electron density estimator by calculating a parameter for the electron density estimator, the parameter minimizing a difference between the first electron density and the second electron density;

(a5) obtaining second input data from a test database in which the material information is stored, and inputting the second input data to the electron density estimator, in which the parameter is set, to estimate a third electron density, and

(a6) outputting the estimated third electron density.

With the present configuration, the first input data and the first electron density are set as the initial values, and the numerical simulation is performed in which the electron density update processing using the density functional method is performed one or more times. The electron density estimator is trained such that the difference between the second electron density obtained by this numerical simulation and the first electron density is minimized. The second electron density is not a convergence value obtained using the density functional method. In this manner, with the present configuration, the electron density estimator is trained using the second electron density, which is an update value obtained by the numerical simulation. Thus, the present configuration makes it possible to train the electron density estimator without using a convergence value. Consequently, training data can be prepared without taking massive time and effort, and the cost needed to generate the electron density estimator can be reduced.

In the above-described configuration, the (a5) may be performed after processing from the (a1) to the (a4) is repeated a predetermined number of times.

With the present configuration, since the electron density estimator can be trained while updating the second electron density through the numerical simulation, the numerical simulation and the training of the electron density estimator can be efficiently performed.

In the above-described configuration, in the (a3),

(b1) in a case where the first electron density updated in the (a2) satisfies a predetermined condition, the numerical simulation may be performed to update the second electron density, and a process may be caused to proceed to the (a4), and

(b2) in a case where the first electron density updated in the (a2) does not satisfy the predetermined condition, the process may be caused to proceed to the (a4) without updating the second electron density.

With the present configuration, the second electron density is not updated every time the first electron density is updated but is updated when the first electron density approaches the second electron density to some extent. Thus, the number of times the numerical simulation, which is high in processing cost, is performed can be reduced, and the electron density estimator can be efficiently trained.

In the above-described configuration, the predetermined condition may be that a second difference between the first electron density updated in the (a2) and a fourth electron density becomes greater than a third difference between the first electron density updated in the (a2) and a fifth electron density,

the fourth electron density may be the first electron density used for calculating the second electron density updated in the (b1), and

the fifth electron density may be the second electron density updated in the (b1).

With the present configuration, the second electron density can be updated on the condition that the updated first electron density has become closer to the fifth electron density, which is the updated second electron density, than to the fourth electron density, which is the first electron density used to update the second electron density. Thus, the number of times the numerical simulation is performed can be reduced, and the electron density estimator can be efficiently trained.

In the above-described configuration, the material information may be information described using Crystallographic Information File.

With the present configuration, the material information is described in an existing data format, and thus the first input data can be easily implemented into the computer.

In the above-described configuration, in the (a4), the electron density estimator may be trained using the second electron density as teacher data.

With the present configuration, the electron density estimator can be trained such that the first electron density approaches the second electron density.

In the above-described configuration, in the (a6), an image indicating the third electron density may be displayed on a display.

The present configuration makes it possible for a user to visually recognize the third electron density.

The operational effects of the above-described electron density estimation method are also realized in substantially the same manner by the electron density estimation apparatus and a recording medium.

### First Embodiment

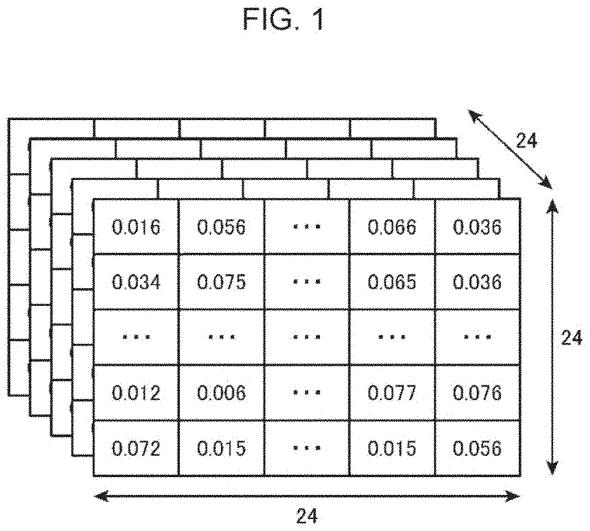

First, reasons why an electron density estimation apparatus of the present disclosure can train an electron density estimator without using an electron density convergence value obtained by a density functional method will be described. FIG. 1 is a diagram illustrating an example of specific electron density values. In a case where the density of electrons in a unit cell of a material is obtained using a density functional method, the unit cell is divided into 24×24×24 three-dimensional cells, and each cell holds a real number indicating an electron density. In this case, the unit cell is, for example, a body-centered cubic lattice or a face-centered cubic lattice, and such a unit cell is divided into 24×24×24 three-dimensional cells. In a case where the density of electrons in a certain space is obtained, individual points in the space are each considered to be represented by a cell, each cell is considered to hold an electrical charge, and the magnitude of the electron density of each cell is obtained. Note that the number of cells for each dimension when the unit cell is divided is not limited to 24, and an arbitrary value other than 24 may be employed. In addition, the number of cells for each dimension when the unit cell is divided may differ from dimension to dimension.

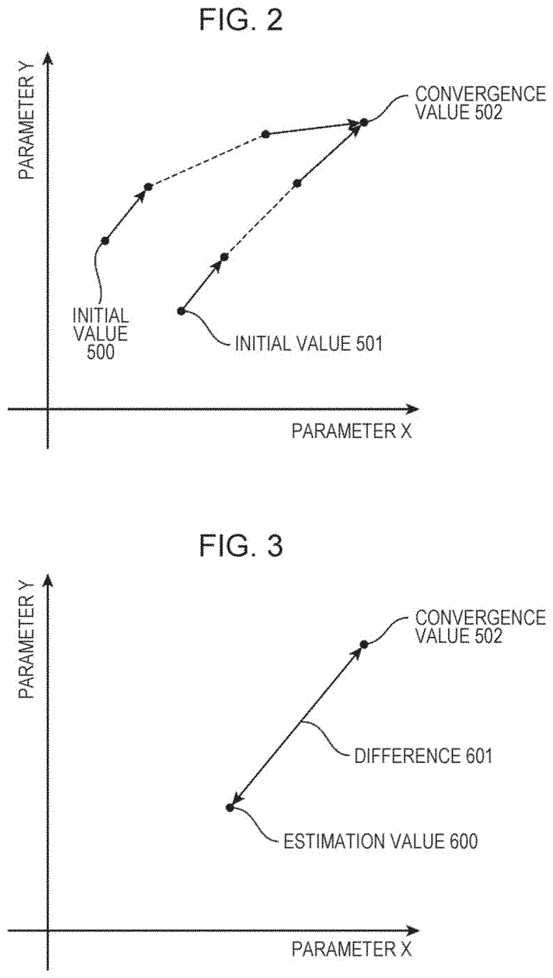

FIG. 2 is a diagram schematically illustrating the way in which the density of electrons is updated by repeating an electron density update calculation in a density functional method. In FIG. 2, the vertical axis represents a parameter Y, and the horizontal axis represents a parameter X. FIG. 2 illustrates the way in which the electron densities of two certain cells among the above-described 24×24×24 cells are updated by repeating the electron density update calculation. The parameter X represents the electron density of one of the two cells, and the parameter Y represents the electron density of the other one of the two cells. Thus, individual points in the graph in FIG. 2 are each constituted by a set of the electron densities of the two cells. Note that since the number of cells is 24×24×24 in the example in FIG. 1, in this case, each point in the graph in FIG. 2 is constituted by a set of 24×24×24 electron densities. The same applies to FIGS. 3 to 5.

An initial value 500 and an initial value 501 each represent an initial electron density in a density functional method. A convergence value 502 represents a final electron density obtained by the density functional method. That is, the convergence value 502 is a value to which the density of electrons is caused to converge by repeating the electron density update calculation in the density functional method. As illustrated in FIG. 2, even when different electron density initial values are used in a case where the electron density update calculation is repeated, the density functional method can theoretically make the density of electrons converge from the initial values 500 and 501 to the same convergence value 502. Note that, the technique illustrated in FIG. 2 is a technique in which the density of electrons is caused to converge by repeating the electron density update calculation in the density functional method, that is, the convergence value 502 is calculated by the density functional method, and thus corresponds to the technique described in NPL 1.

FIG. 3 is a diagram illustrating a difference calculated when an electron density estimator is trained. An estimation value 600 represents an output from the electron density estimator. A difference 601 is the difference between the convergence value 502 and the estimation value 600. To calculate the difference, for example, a root-mean-square error (RMSE) is used. At the time of learning, a parameter for the electron density estimator is updated such that the difference 601 between the estimation value 600 and the convergence value 502 is minimized, that is, the estimation value 600 approaches the convergence value 502. Note that the technique illustrated in FIG. 3 is a technique in which the convergence value 502 for the electron density estimator is calculated by the density functional method and corresponds to the techniques described in NPLs 2 and 3, and thus differs from the techniques described in the present disclosure.

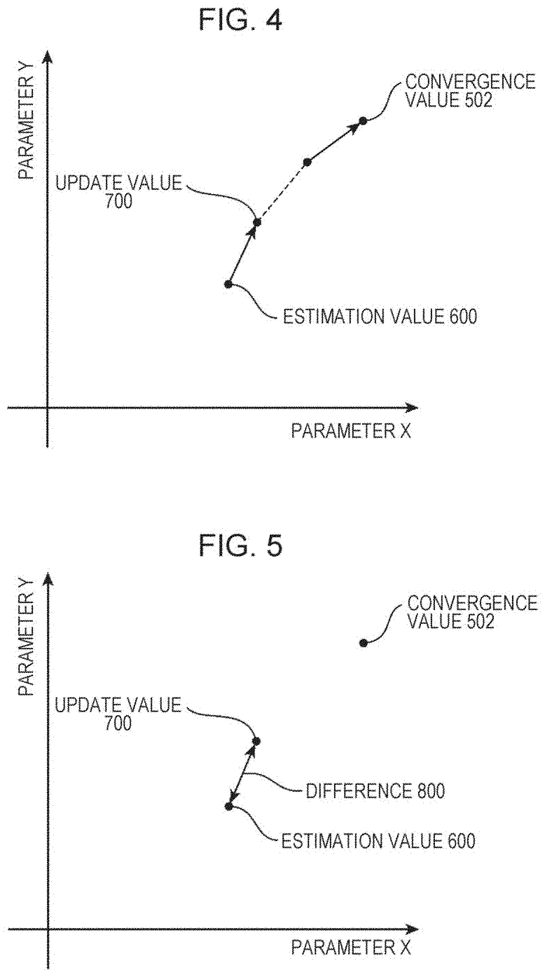

FIG. 4 is a diagram schematically illustrating the way in which the estimation value 600 obtained by the electron density estimator is set as an initial electron density in the density functional method and the density of electrons is updated by repeating an electron density update calculation. An update value 700 is an electron density obtained by performing the electron density update calculation one time in the density functional method using the estimation value 600.

As illustrated in FIG. 2, in the density functional method, even in a case where the different initial values 500 and 501 are used, the density of electrons can be made to converge from the initial values 500 and 501 to the same convergence value 502. Thus, even when the estimation value 600 illustrated in FIG. 4 is used as the initial electron density in the density functional method, the density of electrons can be made to converge from the estimation value 600 to the convergence value 502 by repeating the electron density update calculation in the density functional method. The technique illustrated in FIG. 4 is a technique in which the estimation value 600 obtained by the electron density estimator is used as the initial value electron density in the density functional method, and the convergence value 502 is obtained by repeating the electron density update calculation in the density functional method. Thus, the technique illustrated in FIG. 4 differs from the techniques described in the present disclosure in that the convergence value 502 is calculated by the density functional method.

FIG. 5 is a schematic diagram illustrating the training of an electron density estimator using the update value 700 as teacher data, the update value 700 being obtained by performing an electron density update calculation one time in the density functional method using the estimation value 600. The update value 700 is an electron density obtained by performing the electron density update calculation one time in the density functional method, and is not an electron density convergence value obtained by the density functional method. Note that FIG. 5 corresponds to a technique described in the present disclosure. A difference 800 is the difference between the update value 700 and the estimation value 600. By updating a parameter for the electron density estimator so as to minimize the difference 800, the electron density estimator can be trained such that the estimation value 600 approaches the update value 700.

The update value 700 is a value that causes the estimation value 600 to be updated toward the convergence value 502 by the density functional method. By training the electron density estimator such that the estimation value 600 approaches the update value 700, the electron density estimator becomes able to output an estimation value closer to the convergence value 502.

In this manner, in the technique illustrated in FIG. 5 unlike in the techniques illustrated in FIGS. 2 to 4, even though the density functional method is used, the density functional method does not calculate the convergence value 502 but just calculates the update value 700. The electron density estimator is trained using the update value 700 as teacher data such that the estimation value 600 gradually approaches the convergence value 502 while the electron density update calculation is repeated in the density functional method. Thus, the electron density estimator can be trained without using the convergence value 502. As a result, the number of times the electron density update calculation is performed in the density functional method is reduced, and thus the electron density estimator can be generated that can accurately calculate the density of electrons while reducing the computational cost.

Next, compared with the technique described in a comparative example in which an electron density estimator is trained using a convergence value obtained by a density functional method, the reason why the time needed until the training of an estimator is completed is reduced with the techniques described in the present disclosure will be described using equations.

### Necessary Time Tα of Comparative Example

First, a necessary time Tα of the comparative example will be described. In this case, the number of pieces of training data is denoted by P, the number of times one piece of training data is used at the time of learning is denoted by Q, and the number of times an electron density update calculation is performed in a case where a convergence value is obtained by the density functional method is denoted by R. In addition, the time needed to perform the electron density update calculation one time is denoted by T, and the time needed to train the electron density estimator per piece of training data is denoted by L. In this case, the convergence value obtained by the density functional method is prepared as training data, and the necessary time Ta, which is needed to train the electron density estimator thereafter, is calculated as follows. That is, in this comparative example, P pieces of training data are acquired by performing the electron density update calculation R times for each of P types of material by using the density functional method, and the electron density estimator is trained Q times for each of the P pieces of training data.

Necessary Time Tα=T×R×P+L×Q×P

The first term T×R×P on the right side of the equation is the time needed to perform the density functional method. Since the time needed to perform the electron density update calculation one time is denoted by T and the number of times the electron density update calculation is performed is denoted by R, the time needed to calculate for one piece of training data is T×R. Thus, the time needed to calculate for all the P pieces of training data is T×R×P.

In the density functional method, the density of electrons is calculated by solving the Kohn-Sham equations described in Eqs. (1) and (2) below. Note that Eqs. (1) and (2) are described in Eqs. (2.8) and (2.9) in NPL 1.

$$\begin{matrix}\{ - \frac{1}{2} {∇}^{2} + [ ϕ ( r ) + {μ}_{xc} ( n ( r ) ) ] \} {ψ}_{i} ( r ) = {ϵ}_{i} {ψ}_{i} ( r ) & ( 1 ) \\ n ( r ) = {\sum }_{i = 1}^{N} {\vert {ψ}_{i} ( r ) \vert}^{2} & ( 2 )\end{matrix}$$

The left side of Eq. (1) is defined on the basis of the atomic arrangement and periodic structure of a material to be a calculation target, and a wave function ψᵢ(r) is obtained by solving Eq. (1). Note that

φ(r)

and μ_{xc}(n(r)) on the left side of Eq. (1) are each a function corresponding to the material. In addition, εᵢ on the right side of Eq. (1) is orbital energy corresponding to the wave function ψᵢ(r) and is obtained by solving the equation expressed by Eq. (1).

An electron density n(r) is calculated from the wave function ψᵢ(r) from Eq. (2). However, the electron density n(r) is necessary to define the left side of Eq. (1), and Eq. (1) cannot be solved as is. Thus, the electron density n(r) is set to an appropriate initial value, the set electron density initial value is substituted into Eq. (1), and the electron density is updated by solving Eqs. (1) and (2). The left side of Eq. (1) is defined again using the updated electron density, and the electron density is updated. This electron density update calculation is repeated until the electron density n(r) converges, and the electron density corresponding to the atomic arrangement and periodic structure of the material, which is a calculation target, is calculated.

A “calculation for updating the electron density one time in the density functional method” means that “ψᵢ(r) is obtained from a known electron density and Eq. (1), and a new electron density is calculated from the obtained ψᵢ(r) and Eq. (2)”, that is, “a new electron density is obtained by using the known electron density and using Eq. (1) one time and Eq. (2) one time”.

The second term L×Q×P of the necessary time Tα is the time needed to train the electron density estimator. In the training, since the number of times one piece of training data is used is denoted by Q, and the time needed to learn one piece of training data is denoted by L, the time needed to learn one piece of training data is L×Q. Thus, the time needed to learn P pieces of training data is L×Q×P.

### Necessary Time Tβ According to Technique of Present Disclosure

In the following technique of the present disclosure, a set of processes is repeated Q times, the set of processes being constituted by a process in which an electron density update calculation in a density functional method is performed one time for each of the P types of material to acquire P pieces of training data and a process in which an estimator is trained one time for each of the P training data. That is, per piece of training data, the electron density update calculation is performed one time and the training processing is performed one time, and thus R, which is the number of times the electron density update calculation is performed, becomes equal to Q, which is the number of times one piece of training data is used.

Thus, a necessary time Tβ according to the technique described in the present disclosure corresponds to the necessary time Tα obtained when R=Q and is calculated as below.

Necessary time Tβ=(T+L)×Q×P

In this case, when the electron density estimator learns one piece of training data, the electron density update calculation in the density functional method is performed one time. Thus, the time needed for the electron density estimator to learn one piece of training data is (T+L), which is the sum of the time T needed to perform the electron density update calculation one time and the time L needed to learn one piece of training data. Thus, the time needed when each of the P pieces of training data is learned Q times is (T+L)×Q×P.

### Comparison Between Necessary Time Tα and Necessary Time Tβ

When the necessary time Tβ is subtracted from the necessary time Tα, the following expression is obtained.

T×(R−Q)×P

Thus, in a case where R, which is the number of times the electron density update calculation in the density functional method is performed, is greater than Q, which is the number of times one piece of training data for the electron density estimator is used, T×(R−Q)×P becomes positive, and the necessary time Tβ of the present disclosure becomes shorter than the necessary time Tα of the comparative example.

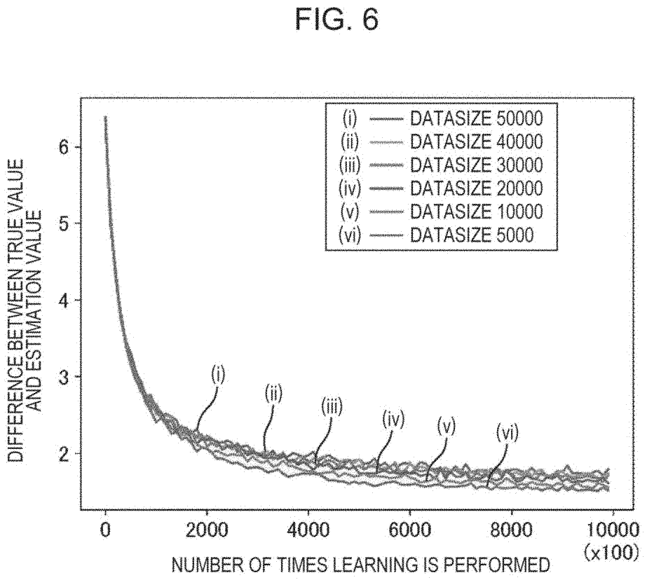

FIG. 6 is a diagram illustrating the progress of learning in a case where the number of pieces of training data is changed for a classification problem using a natural image data set called the CIFAR-10 data set. In FIG. 6, the vertical axis represents the difference between a true value and an estimation value, and in a case where this difference has converged, the training of the electron density estimator ends. The horizontal axis represents the number of times learning is performed. FIG. 6 illustrates the way in which the difference is converging using six graphs corresponding to data sizes of 5,000, 10,000, 20,000, 30,000, 40,000, and 50,000. In this case, each data size represents the number of pieces of training data.

As illustrated in FIG. 6, even when the data size is changed from 5,000 to 50,000 in learning, there is no big difference between the numbers of times learning needs to be performed until the difference converges. For example, in the case of learning in which the data size is 50,000, assume that the difference has converged after learning is performed 10,000×100 times. In this case, the number of times learning is performed is represented by the product of Q and P, Q being the number of times one piece of training data is used, P being the number of pieces of training data, and thus the number of times one piece of training data is used, Q, is calculated as 20 from the following equation.

Q=10,000×100/P=20

In addition, even when the number of pieces of training data, P, is increased, the number of times learning needs to be performed to realize the convergence does not increase much. Thus, in the equation above, the number of times learning needs to be performed to realize the convergence (10,000×100) can be considered to be constant. Thus, in the equation above, in a case where the number of pieces of training data, P, is increased, the number of times one piece of training data is used, Q, to realize the convergence can be reduced.

In contrast, R, which is the number of times the electron density update calculation is performed, needs to be about 40 on average although the number of times the electron density update calculation needs to performed depends on a material to be a calculation target. In addition, R, which is the number of times the electron density update calculation is performed, needs to be increased as the accuracy of calculation is increased.

From the above, in a case where at least 50,000 or more pieces of training data are used, since R=40 and Q=20, T×(R−Q)×P becomes positive. Thus, the technique described in the present disclosure can reduce the necessary time more greatly than that described in the comparative example.

### First Embodiment: Description of Configuration

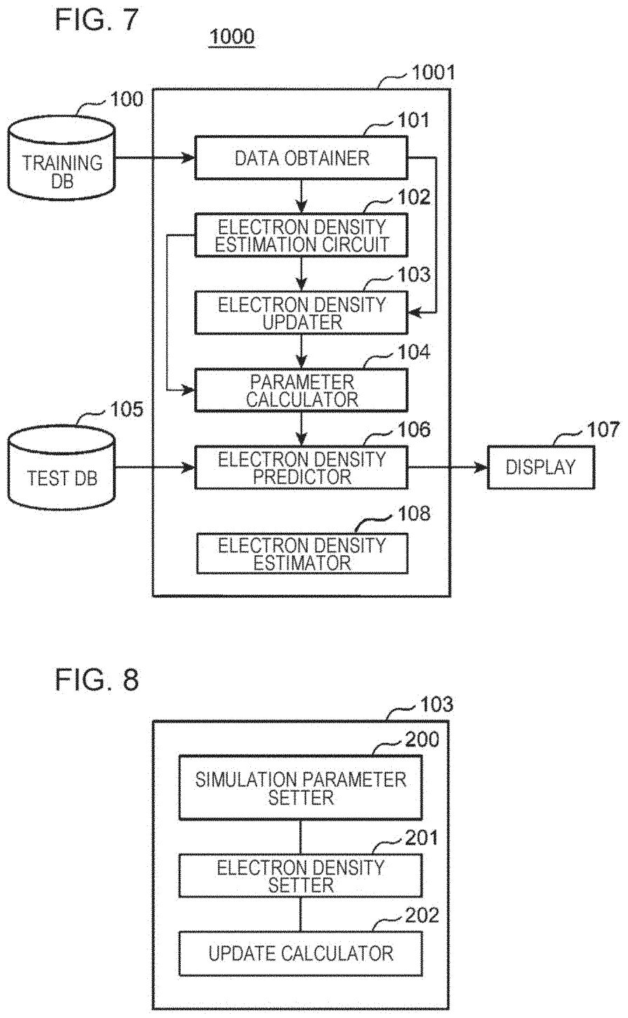

Next, the configuration of the first embodiment will be described. In the following, an electron density estimation system 1000 according to the first embodiment of the present disclosure will be described in detail using the drawings. FIG. 7 is a block diagram illustrating the configuration of the electron density estimation system 1000 according to an embodiment of the present disclosure. The electron density estimation system 1000 illustrated in FIG. 7 includes an electron density estimation apparatus 1001, a training database (DB) 100, a test database (DB) 105, and a display 107.

The electron density estimation apparatus 1001 includes a data obtainer 101, an electron density estimation circuit 102, an electron density updater 103, a parameter calculator 104, an electron density predictor 106, and an electron density estimator 108. Note that, in FIG. 7, the electron density estimation apparatus 1001 is realized by a processor such as a central processing unit (CPU) executing an electron density estimation program, which causes the computer to function as the electron density estimation apparatus 1001. In addition, in FIG. 7, the training DB 100 and the test DB 105 are constituted by a nonvolatile memory.

In the following, the details of each constitutional element illustrated in FIG. 7 will be described.

### Training DB 100

The training DB 100 prestores, regarding one or more materials, first input data including material information such as the composition and structure of each of the one or more materials. The first input data includes, for example, material information described in Crystallographic Information File (CIF). Note that the description format of the material information is not limited to the CIF data format and may also be any description format as long as the electron density update calculation in the density functional method can be performed. Examples of such a description format include a composition formula, a crystal structure, a space group, and a lattice vector.

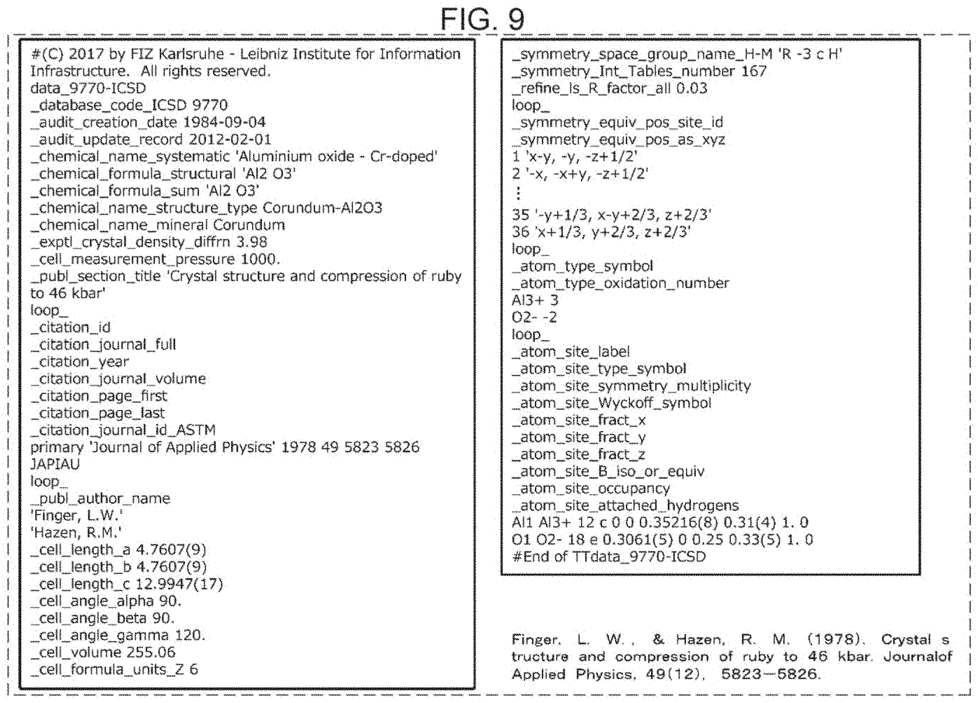

FIG. 9 is a diagram illustrating an example of the first input data described in a format called CIF. In CIF, material information is described that includes, for example, a composition formula, the length of a unit cell vector, the angle at which atoms meet with each other, and the atomic arrangement in a unit cell. In FIG. 9, material information regarding a material “Al₂O₃” is illustrated.

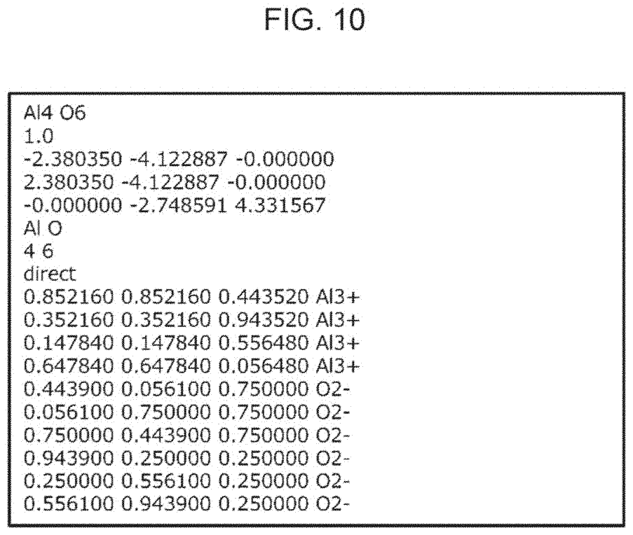

FIG. 10 is a diagram illustrating an example of the first input data described in a format called POSCAR. In POSCAR, material information is described that includes, for example, a composition formula, a unit cell vector, and the atomic arrangement in a unit cell. Note that the description illustrated in FIG. 9 is cited from some documents (see NPL 4 and “Bergerhoff, G., Brown, I. D., & Allen, F. (1987). Crystallographic databases. International Union of Crystallography, Chester, 360, 77-95”).

### Data Obtainer 101

Reference is made again to FIG. 7. The data obtainer 101 obtains the first input data from the training DB 100 and outputs the first input data to the electron density estimation circuit 102 and the electron density updater 103.

### Electron Density Estimation Circuit 102

The electron density estimation circuit 102 obtains the first input data from the data obtainer 101 and calculates a descriptor from the first input data. The electron density estimation circuit 102 sends the calculated descriptor to the electron density estimator 108. The electron density estimator 108 inputs the descriptor to an input layer of the electron density estimator 108, calculates a first electron density, and outputs the first electron density from an output layer of the electron density estimator 108. The electron density estimator 108 sends the first electron density to the electron density estimation circuit 102. The electron density estimation circuit 102 outputs the first electron density to the electron density updater 103 and the parameter calculator 104.

The descriptor is expressed by a vector or a two-dimensional or three-dimensional matrix that can be calculated from the first input data. The descriptor may also be expressed by a combination of first input data of constituent elements and physical property values of the elements. Alternatively, the descriptor may include a potential as illustrated in FIG. 1 of NPL 2 and FIG. 1 of NPL 3. Alternatively, the descriptor may include the initial electron density in the density functional method.

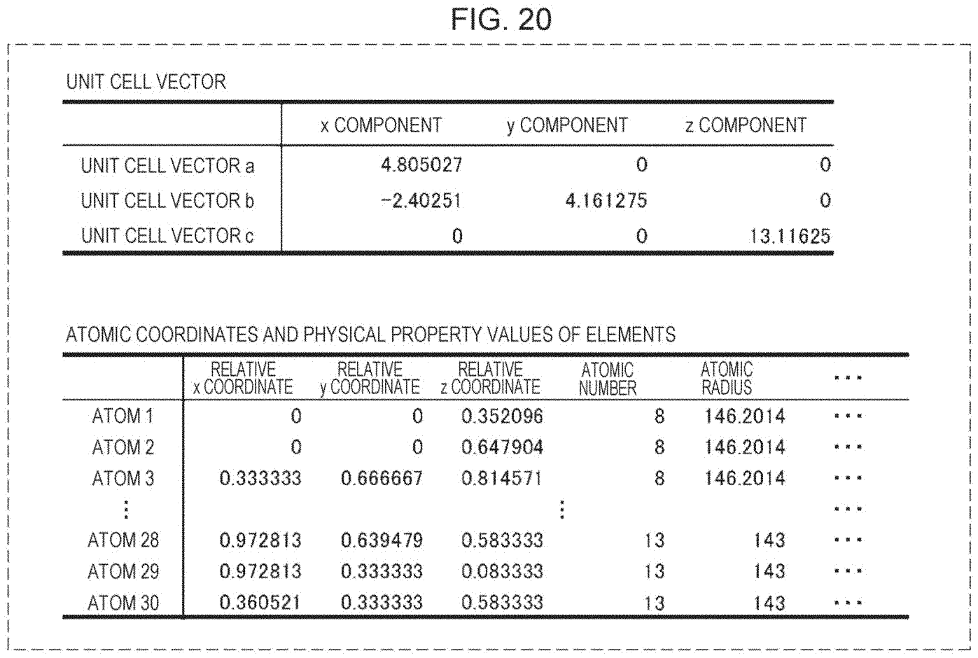

For Al₂O₃, an example of the descriptor expressed by a combination of the first input data and physical property values of the elements will be described below. FIG. 20 is a diagram illustrating unit cell vectors, atomic coordinates, atomic numbers, atomic radii, and the like regarding Al₂O₃. The descriptor of Al₂O₃ is a one-dimensional vector in which the unit cell vectors of Al₂O₃, the relative coordinates of Al atoms, the relative coordinates of O atoms, and the atomic number and atomic radius of each of Al and O are listed.

For example, (Al₂O₃ descriptor)=(4.805027 0 0−2.40251 4.161275 0 0 0 13.11625 0 0 0.352096 8 146.2014 0 0 0.647904 8 146.2014 . . . ). The listing order of the elements included in the vector may be arbitrarily determined.

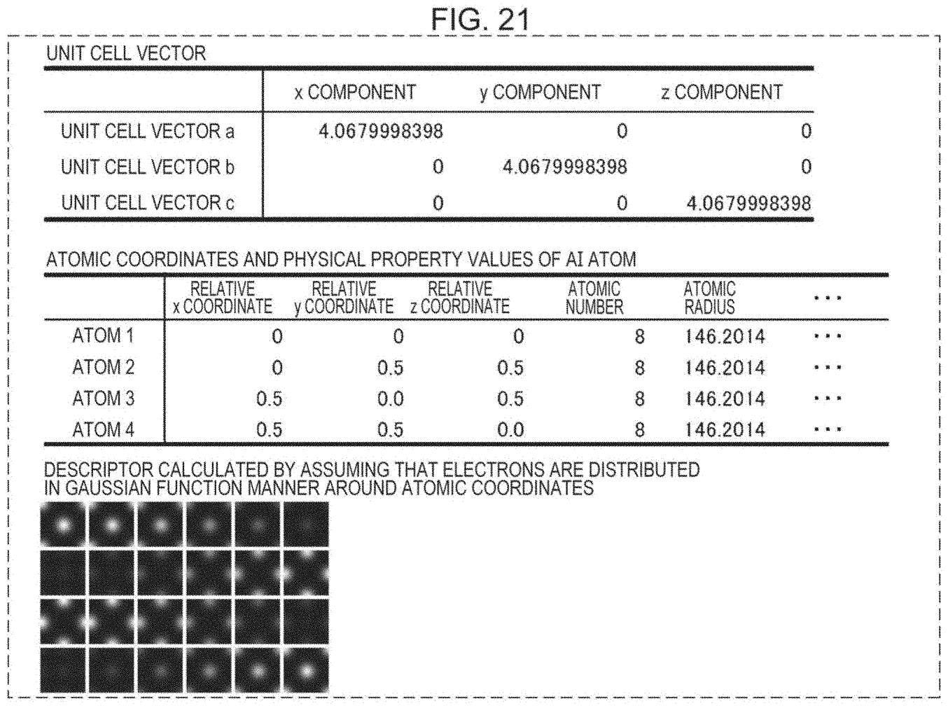

FIG. 21 is a diagram illustrating, for example, an example of a descriptor of an Al crystal expressed by a combination of first input data and the atomic number or the atomic radius. For example, in the case of an Al crystal, the density of electrons is considered to exist in a Gaussian distribution manner around the atomic coordinates of a face-centered cubic lattice, and the values of 24×24×24 cells are calculated as expressed by the following equation.

$$The value of each cell = {\sum }_{d < {d}^{′}} \frac{α}{\sqrt{2 πσ}} exp ( - \frac{{d}^{2}}{2 {σ}^{2}} ) .$$

Note that d=the distance between the cell and the atom, α=the atomic number, σ=the atomic radius/3, and d′=a threshold.

For each cell, the value that attenuates in accordance with the distance between the center point of the cell and each atom is multiplied by the atomic number of the atom, and the values obtained for atoms that are included in a predetermined radius are summed up. In this case, for σ of the Gaussian distribution, it is sufficient that the atomic radius/3 be used. For the predetermined radius, for example, it is sufficient that the value of the longest one of the diagonal lines of a unit cell be used. Note that, instead of the atomic number, the electronegativity or ionization energy of each atom may also be used.

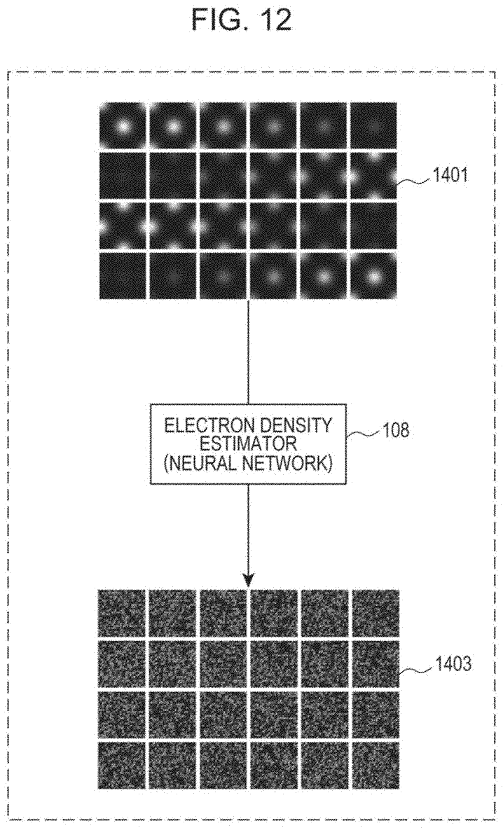

FIG. 12 is a diagram illustrating an example of the initial electron density, the first electron density, and the electron density estimator 108.

FIG. 1 illustrates the densities of electrons of 24×24×24 cells. It may be considered that FIG. 1 illustrates 24 images and each of the 24 images illustrates 24×24 pixels. It may also be considered that each of the 24×24 pixels has a pixel value and the pixel value of one pixel corresponds to the density of electrons of one cell.

The number of units of the input layer of the electron density estimator 108 in FIG. 12 may be 24×24×24. The pixel values of the pixels of the 24 images (the number of pixels=24×24×24) illustrated as an initial electron density 1401 in FIG. 12 are input to the units of the input layer of the electron density estimator 108. The initial electron density 1401 is a descriptor generated by the electron density estimation circuit 102.

The pixels of the 24 images illustrated as the initial electron density 1401 correspond to the units of the input layer of the electron density estimator 108 in a one-to-one manner.

The number of units of the output layer of the electron density estimator 108 in FIG. 12 may be 24×24×24. The pixel values of the pixels of the 24 images (the number of pixels=24×24×24) illustrated as a first electron density 1403 in FIG. 12 are output from the units of the output layer of the electron density estimator 108.

The pixels of the 24 images illustrated as the first electron density 1403 correspond to the units of the output layer of the electron density estimator 108 in a one-to-one manner.

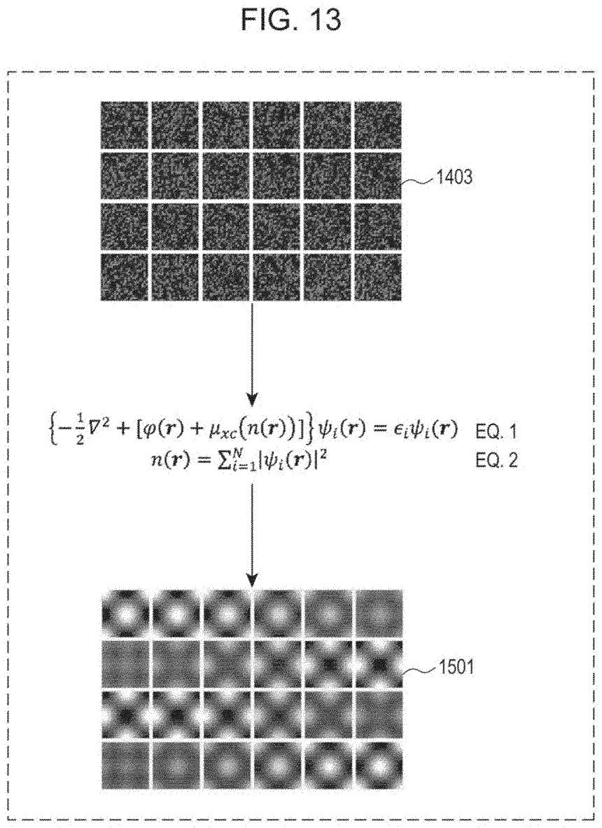

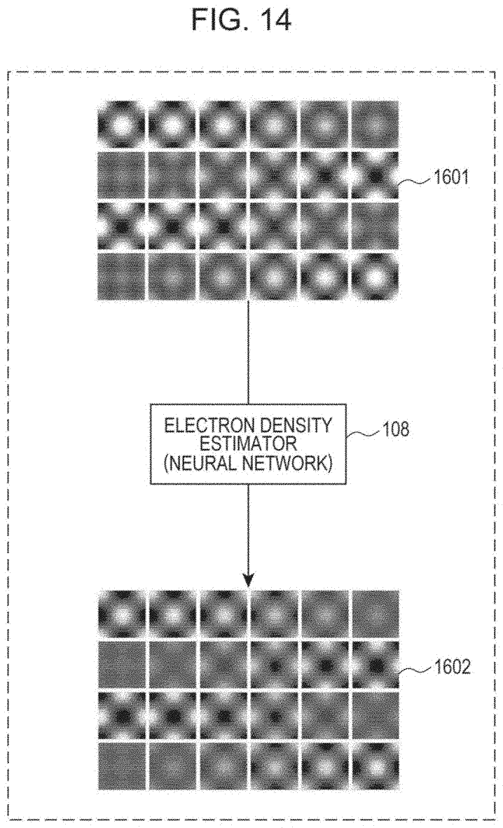

In FIG. 12, white portions indicate portions having a high density of electrons. The same applies to FIGS. 13 and 14 described later. The initial electron density 1401 includes densities of electrons obtained in a case where it is assumed that electrons exist near a nucleus. In this case, as an example, the initial electron density 1401 for an Al monocrystal is illustrated in a case where the densities of electrons of the 24×24×24 cells are expressed by 24 images each having 24×24 cells.

### Electron Density Estimator 108

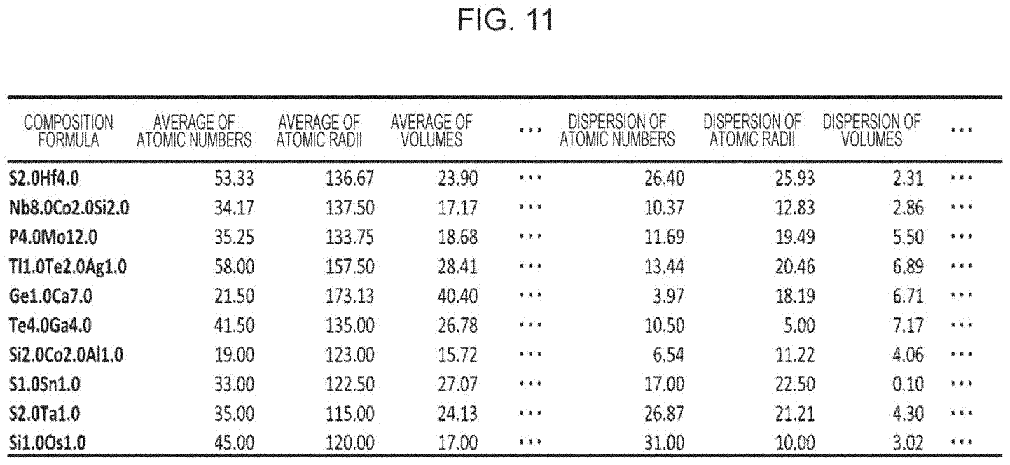

The electron density estimator 108 receives the initial electron density 1401 (the descriptor generated by the electron density estimation circuit 102) and outputs the first electron density 1403. In this example, the electron density estimator 108 may be constituted by the neural network illustrated in FIG. 2 of NPL 5. Note that the electron density estimator 108 may be constituted by a random forest or a support vector machine that learns a (24×24×24)-dimensional regression problem. Alternatively, a set of the descriptor in FIG. 11 and the initial electron density may be input to the electron density estimator 108. The electron density estimation apparatus 1001 does not have to include the electron density estimator 108, and an external server may include the electron density estimator 108. In this case, the electron density estimation apparatus 1001 and the external server may send and receive data to and from each other in a wired manner, a wireless manner, or a wired and wireless manner.

### Electron Density Updater 103

Reference is made again to FIG. 7. The electron density updater 103 obtains the first input data from the data obtainer 101 and the first electron density from the electron density estimation circuit 102, performs a numerical simulation using the first input data and the first electron density to calculate a second electron density, and outputs the second electron density to the parameter calculator 104.

The numerical simulation is processing in which the electron density update calculation in the density functional method is performed one time using the first input data and the first electron density. The second electron density is an electron density updated by the numerical simulation.

Note that the numerical simulation may be performed one or more times. That is, first, “the electron density update calculation in the density functional method is performed one time using the first input data and the first electron density to obtain a first provisional electron density”. Next, “the electron density update calculation in the density functional method is performed one time using the first input data and the first provisional electron density to obtain a second provisional electron density”. Such a calculation is repeated, and lastly “the electron density update calculation in the density functional method is performed one time using the first input data and an (n−1)-th provisional electron density to obtain an n-th provisional electron density”. The n-th provisional electron density may be treated as the second electron density described above. The number of times the electron density update calculation is performed is not the number of update calculations performed to obtain an electron density limit in the density functional method. Note that n may be less than or equal to five.

By performing the numerical simulation one or more times, the stability of the result obtained by the numerical simulation can be improved. In a general density functional method, the numerical simulation is stabilized by recording the last five densities of electrons or so in the update history and mixing the previous densities of electrons and the post-update density of electrons at an appropriate ratio. Even in the technique described in the present disclosure, the numerical simulation can be stabilized by performing the numerical simulation one or more times (five times at maximum) when the density of electrons is updated.

Note that, in a case where the numerical simulation is performed five times and where at least 250,000 or more pieces of training data are used, the technique described in the present disclosure can reduce the necessary time more greatly than the technique described in the comparative example.

The number of times the numerical simulation is performed may be the number of times after which the result obtained from the numerical simulation can be stabilized and it is possible to complete the numerical simulation in a time period shorter than the time period needed to obtain a limit through the numerical simulation.

FIG. 13 is a diagram illustrating an example of processing in which the second electron density is calculated from the first electron density. The electron density updater 103 substitutes the first electron density 1403 into Eq. (1) and also

φ(r)

and μ_{xc}(n(r)) obtained from the first input data into Eq. (1) to obtain the wave function ψᵢ(r). The electron density updater 103 substitutes the obtained wave function ψᵢ(r) into Eq. (2), and calculates the obtained electron density n(r) as a second electron density 1501. Eq. (1) includes the atomic coordinates, and thus even when random electron coordinates such as the first electron density 1403 are set as an initial value, the electron density in which the atomic coordinates are reflected such as the second electron density 1501 can be theoretically obtained by calculating a Kohn-Sham equation.

### Parameter Calculator 104

Reference is made again to FIG. 7. The parameter calculator 104 obtains the first electron density from the electron density estimation circuit 102 and obtains the second electron density from the electron density updater 103. The parameter calculator 104 calculates a first difference, which is the difference between the first electron density and the second electron density, calculates a parameter for the electron density estimator, the parameter minimizing the first difference, and outputs the calculated parameter to the electron density predictor 106.

As the first difference, the RMSE described above can be used. Note that this is an example, and the first difference may be calculated using any method as long as the method can evaluate errors such as the mean absolute error (MAE). To calculate the parameter that minimizes the first difference, for example, backpropagation can be used. A parameter update equation for backpropagation is, for example, expressed by the following equation.

W_t+1=W_t−γdD(W_t)/dW_t,

where W_t denotes a pre-update parameter value, W_t+1 denotes a post-update parameter value, D(W_t) denotes the first difference, and γ denotes a learning coefficient. The value of the learning coefficient is not particularly limited, however, for example, 0.01 can be used.

To update the parameter W_t in accordance with the update equation described above, all the first input data stored in the training DB 100 may be used.

Alternatively, the parameter calculator 104 obtains a portion of the first input data from the training DB 100, updates the parameter W_t about the obtained portion of the first input data by using the update equation described above, and thereafter obtains another portion of the first input data from the training DB 100. In this manner, the parameter W_t may be repeatedly updated by sequentially obtaining a different portion of the first input data from the training DB 100.

The parameter calculator 104 calculates, for the electron density estimator 108, the parameter that minimizes the first difference, and the calculation of this parameter can be considered as execution of the following processes (P1) to (P4) and specification of a k-th parameter.

### Process (P1)

The descriptor generated by the electron density estimation circuit 102 on the basis of the first input data is input to the electron density estimator 108, in which a first parameter is set. The electron density estimator 108 calculates and outputs a first electron density D11.

### Process (P2)

The electron density updater 103 performs the electron density update calculation using the density functional method one or more times to calculate the second electron density. This calculation performed one or more times includes a calculation using the density functional method based on the first input data and the first electron density D11. The second electron density is not a convergence value obtained using the density functional method.

### Process (P3)

The generated descriptor described above is input to the electron density estimator 108, in which an i-th parameter is set. The electron density estimator 108 calculates and outputs a first electron density D1i. In this case, i=2 to n, i is a natural number, and n is a natural number greater than or equal to 2. Consequently, first electron densities D12 to D1n are obtained.

### Process (P4)

The parameter calculator 104 calculates a first difference DF1j, which is the difference between the first electron density D1j and the second electron density, where j is a natural number. Consequently, first differences DF11 to DF1n are obtained. The first parameter corresponds to the first difference DF11, . . . , and an n-th parameter corresponds to the first difference DF1n.

The parameter calculator 104 detects the smallest first difference DF1k among the first differences DF11 to DF1n and specifies a k-th parameter corresponding to the first difference DF1k, where 1≤k≤n, and k is a natural number. Here, the description of the processes (P1) to (P4) is completed.

The parameters may also be those described below.

The electron density estimator 108 may include the input layer, one or more hidden layers, and the output layer. Each of the input layer and the one or more hidden layers has units corresponding to neurons. The output layer has one or more units.

For each of the units of the one or more hidden layers and the output layer, a weight value Wi=[wi1, wi2, . . . ] is set. That is, in a case where the total number of units of the one or more hidden layers and the output layer is z, there are a weight value W1=[w11, w12, . . . ], . . . , and a weight value Wz=[wz1, wz2, . . . ]. When input data is input to each unit of the input layer, calculation is performed using the weight values W1 to Wz, and output data is output from the one or more units of the output layer.

The first to n-th parameters each have values corresponding to the weight values W1 to Wz. That is, the first parameter has a weight value W1₁=[w11₁, w12₁, . . . ], . . . , and a weight value Wz₁=[wz1₁, wz2₁, . . . ], and the n-th parameter has a weight value W1ₙ=[w11ₙ, w12ₙ, . . . ], . . . , and a weight value Wzₙ=[wz1ₙ, wz2ₙ, . . . ]. The same applies to the other parameters.

### Test DB 105

The test DB 105 prestores, regarding one or more materials, second input data including material information such as the composition and structure of each of the one or more materials. In the second input data, for example, the material information is described in Crystallographic Information File (CIF). Note that the description format of the material information is not limited to the CIF data format and may also be any description format as long as the calculation according to a density functional method is executable. Examples of such a description format include a composition formula, a crystal structure, a space group, and a lattice vector. Note that, similarly to the first input data, the second input data is described so as to have the content as illustrated in FIGS. 9 and 10. In this manner, the second input data and the first input data are described in the same format. Thus, the training DB 100 and the test DB 105 may be configured by a single database. In this case, a portion of input data stored in this database may be used as the first input data, and the rest of the input data may be used as the second input data.

### Electron Density Predictor 106

The electron density predictor 106 obtains a post-update parameter from the parameter calculator 104 and sets the obtained parameter in the electron density estimator 108. The electron density predictor 106 obtains the second input data from the test DB 105 and calculates a descriptor from the obtained second input data. The electron density predictor 106 sends the calculated descriptor to the electron density estimator 108. The electron density estimator 108 inputs the descriptor to the input layer of the electron density estimator 108, calculates a third electron density, and outputs the third electron density from the output layer of the electron density estimator 108. The electron density estimator 108 sends the third electron density to the electron density predictor 106. The electron density predictor 106 outputs the third electron density to the display 107. A calculation method for the descriptor and the third electron density is the same as that for the descriptor and the first electron density in the electron density estimation circuit 102. The calculation of the third electron density may also be called estimation of the third electron density.

FIG. 14 is a diagram illustrating an example of processing in which the third electron density is estimated. An initial electron density 1601 is an electron density obtained as the descriptor for the second input data. A third electron density 1602 is obtained by inputting the initial electron density 1601 to the electron density estimator 108, which has been trained. The localized electron density at the center and four corners of each image is reduced in the third electron density 1602 unlike in the initial electron density 1601, and instead the delocalized electron density appears at positions between the center and four corners. This shows that the electron density is accurately estimated.

### Display 107

The display 107 includes, for example, a display device such as a liquid crystal panel and displays an image indicating a third electron density estimated by the electron density predictor 106. The display 107 is an example of an outputter that outputs the third electron density. As the image indicating the third electron density, for example, the image displaying the third electron density 1602 illustrated in FIG. 14 may be used, or an image displaying a group of numerical values indicating the third electron density may be used.

FIG. 8 is a block diagram illustrating a detailed configuration of the electron density updater 103 in FIG. 7. The electron density updater 103 illustrated in FIG. 8 includes a simulation parameter setter 200, an electron density setter 201, and an update calculator 202.

In the following, the details of each constitutional element illustrated in FIG. 8 will be described.

### Simulation Parameter Setter 200

The simulation parameter setter 200 obtains the first input data from the data obtainer 101 and sets calculation conditions for the numerical simulation. Consequently, for example, functions φ(r) and μ_{xc}(n(r)) of Eq. (1) are set.

### Electron Density Setter 201

The electron density setter 201 obtains the first electron density from the electron density estimation circuit 102 and sets the first electron density as an initial electron density for the numerical simulation. Consequently, the electron density n(r) in Eq. (1) is set.

### Update Calculator 202

The update calculator 202 performs the electron density update calculation in the density functional method one or more times, updates the initial electron density, calculates the second electron density, and outputs the calculated second electron density to the parameter calculator 104. Consequently, the update calculation using Eqs. (1) and (2) is performed one or more times, and the second electron density 1501 is calculated from the first electron density 1403 as illustrated in FIG. 13. Note that the number of times the update calculation is performed is not the number of update calculations performed to obtain an electron density limit in the density functional method.

### First Embodiment: Description of Operation

Next, the operation of the electron density estimation apparatus 1001 will be described.

### Flow Chart

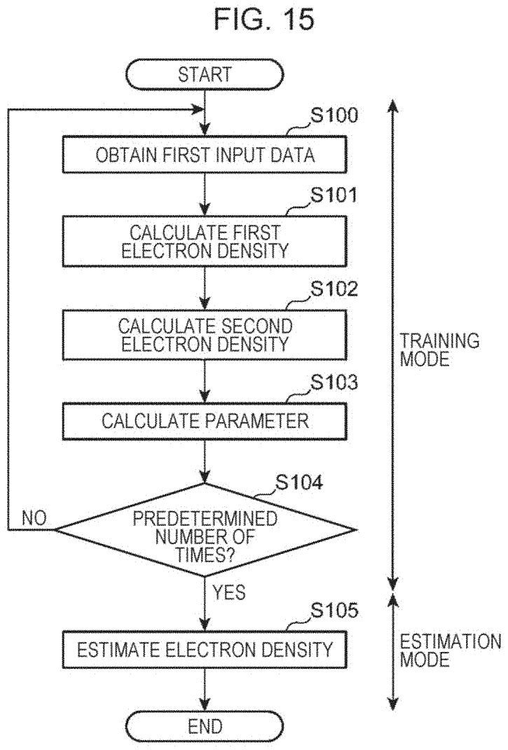

FIG. 15 is a flow chart illustrating the overall procedure of processing performed by the electron density estimation apparatus 1001 in the first embodiment of the present disclosure.

### Step S100

The data obtainer 101 obtains first input data from the training DB 100 and outputs the obtained first input data to the electron density estimation circuit 102 and the electron density updater 103.

### Step S101

The electron density estimation circuit 102 calculates a descriptor from the first input data and sends the calculated descriptor to the electron density estimator 108. The electron density estimator 108 calculates a first electron density and sends the first electron density to the electron density estimation circuit 102. The electron density estimation circuit 102 outputs the first electron density to the electron density updater 103.

### Step S102

The electron density updater 103 performs a numerical simulation according to the density functional method using the first input data obtained in step S100 and the first electron density calculated in step S101 to calculate a second electron density and outputs the second electron density to the parameter calculator 104. In this case, in one loop from step S100 to step S104, the electron density updater 103 performs the calculation of the numerical simulation one time. That is, the electron density update calculation in the density functional method is performed one time. In this case, in a case where the number of loops from step S100 to step S104 is k (k is an integer greater than or equal to one), the total number of times the numerical simulation is performed is k. The total number of times the update calculation is performed, k, is not the number of update calculations performed to obtain an electron density limit in the density functional method. Note that this is an example, and the electron density updater 103 may perform a calculation for the numerical simulation m times (m is greater than one) in one loop. That is, the electron density update calculation in the density functional method may be performed m times. In this case, the total number of times the numerical simulation is performed is k×m. The total number of times the update calculation is performed, k×m, is not the number of update calculations performed to obtain an electron density limit in the density functional method.

### Step S103

The parameter calculator 104 calculates the first difference from the first electron density and the second electron density and updates the parameter W_t for the electron density estimator 108 using, for example, backpropagation such that the first difference is minimized.

### Step S104

In a case where the number of times the parameter has been updated exceeds a predetermined number of times (YES in step S104), the parameter calculator 104 outputs the updated parameter to the electron density predictor 106, and the process proceeds to step S105. Consequently, a trained parameter that minimizes the parameter W_t for the electron density estimator 108 is obtained, and the training of the electron density estimator 108 ends.

In contrast, in a case where the number of times the parameter has been updated does not exceed the predetermined number of times (NO in step S104), the process returns to step S100, and the parameter calculator 104 repeats processing from step S100 to step S103. In this case, as described using FIG. 6, a preset number of updates after which the first difference is expected to converge from the number of pieces of first input data used to train the electron density estimator 108 can be used as the predetermined number of times. Note that this is an example, and processing from step S100 to step S103 may be repeated until the first difference converges to less than or equal to the predetermined value.

### Step S105

The electron density predictor 106 obtains the trained parameter from the parameter calculator 104 and sets the obtained parameter in the electron density estimator 108. The electron density predictor 106 then obtains second input data from the test DB 105, calculates a descriptor from the obtained second input data, and sends the calculated descriptor to the electron density estimator 108. The electron density estimator 108 calculates a third electron density and sends the third electron density to the electron density predictor 106. The electron density predictor 106 outputs the third electron density to the display 107.

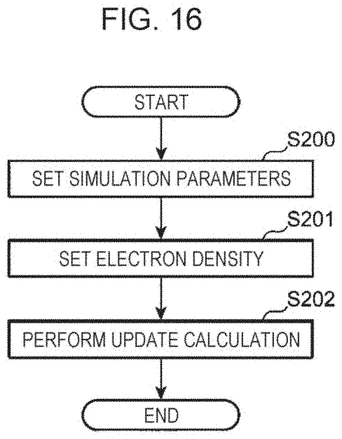

FIG. 16 is a flow chart for describing details of processing performed by the electron density updater 103 in step S102 of FIG. 15.

### Step S200

The simulation parameter setter 200 of the electron density updater 103 sets the first input data obtained from the data obtainer 101 as calculation conditions for the density functional method. In this case, the calculation conditions include φ(r) and μ_{xc}(n(r)) expressed in Eq. (1), and these functions are calculated from the first input data and set in Eq. (1).

### Step S201

The electron density setter 201 sets the first electron density obtained from the electron density estimation circuit 102 as the initial electron density for the density functional method. Consequently, the first electron density obtained from the electron density estimator 108 is set as an initial value of the electron density n(r) of Eq. (1), which makes it possible to solve Eq. (1) and calculate the wave function ψᵢ(r) and orbital energy εᵢ.

### Step S202

The update calculator 202 performs the electron density update calculation in the density functional method one or more times, updates the first electron density, which is set as the initial electron density, to calculate a second electron density, and outputs the second electron density to the parameter calculator 104. The number of times the update calculation is performed is not the number of update calculations performed to obtain an electron density limit in the density functional method.

As above, the electron density updater 103 calculates the second electron density by performing processing from step S200 to step S202.

As described above, according to the present embodiment, the electron density estimator 108 can be trained using the second electron density, which is an update value obtained by performing the electron density update calculation one or more times in the numerical simulation, as training data. The number of times the update calculation is performed is not the number of update calculations performed to obtain an electron density limit in the density functional method. Thus, with the present configuration, the electron density estimator 108 can be trained without using an electron density convergence value obtained by the density functional method. Consequently, training data can be prepared without taking massive time and effort, and the cost needed to generate the electron density estimator 108 can be reduced.

### Second Embodiment

In a second embodiment, the numerical simulation is skipped until the first electron density, to some extent, approaches the second electron density, and the second electron density is updated after the first electron density has, to some extent, approached the second electron density. Note that, in the present embodiment, the same constitutional elements as those of the first embodiment will be denoted by the same reference numerals and the description thereof will be omitted.

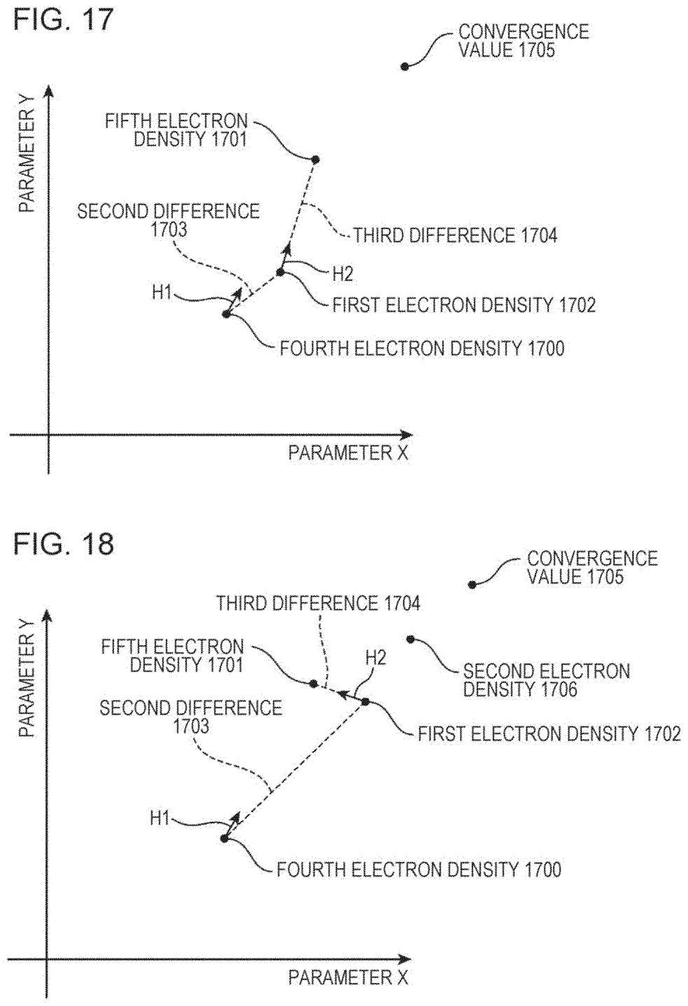

FIG. 17 is a diagram for describing processing in the second embodiment of the present disclosure. FIG. 17 schematically illustrates, in a state in which a second difference 1703 is smaller than a third difference 1704, a distance relationship between a first electron density, a fourth electron density, and a fifth electron density. Note that the vertical axis and the horizontal axis in FIG. 17 are the same as those in FIG. 2.

A fourth electron density 1700 is the first electron density output by the electron density estimator 108 when the last numerical simulation is performed. A first electron density 1702 is a current output from the electron density estimator 108. The second difference 1703 is the difference between the first electron density 1702 and the fourth electron density 1700 and represents changes in the output from the electron density estimator 108 as the training progresses. The third difference 1704 is the difference between a fifth electron density 1701 and the first electron density 1702 and represents, similarly to as in the case of the second difference 1703, changes in the output from the electron density estimator 108 as the training progresses.

The fifth electron density 1701 is a second electron density updated by the numerical simulation in which the fourth electron density 1700 is set as the initial electron density. When viewed from the fourth electron density 1700, the fifth electron density 1701 is positioned on the side where a convergence value 1705 is positioned. Thus, when viewed from the fourth electron density 1700, the fifth electron density 1701 indicates a rough direction toward the convergence value 1705. Thus, by training the electron density estimator 108 using the fifth electron density 1701 as teacher data, the electron density estimator 108 can be trained toward the convergence value 1705. Note that the convergence value 1705 is a value to which the density of electrons is caused to converge by repeating the electron density update calculation in the density functional method.

In the state in which the second difference 1703 is smaller than the third difference 1704, a direction H1 from the fourth electron density 1700 to the fifth electron density 1701 roughly matches a direction H2 from the first electron density 1702 to the fifth electron density 1701. Thus, in this state, the first electron density 1702, which is an output from the electron density estimator 108, can be made to approach the convergence value 1705 by updating the parameter for the electron density estimator 108 such that the third difference 1704 is minimized. In this case, since the numerical simulation is skipped in the present embodiment, the calculation time can be reduced.

FIG. 18 is a diagram for describing processing in the second embodiment of the present disclosure. FIG. 18 schematically illustrates a distance relationship between the first electron density, the fourth electron density, and the fifth electron density in a case where the training of the electron density estimator 108 is continued without performing the numerical simulation even in a state in which the second difference 1703 is greater than the third difference 1704 in FIG. 17. FIG. 18 illustrates a comparative example of the present disclosure. Note that the vertical axis and the horizontal axis in FIG. 18 are the same as those in FIG. 2.

As illustrated in FIG. 18, when the training of the electron density estimator 108 is continued using the fifth electron density 1701 as teacher data without updating the second electron density through the numerical simulation even in the state in which the second difference 1703 is greater than the third difference 1704, it is no longer possible to say that the direction H2 roughly matches the direction H1.

Thus, in the present embodiment, when the second difference 1703 becomes greater than the third difference 1704, that is, the first electron density 1702 becomes closer to the fifth electron density 1701 than to the fourth electron density 1700, the parameter calculator 104 performs the numerical simulation. Consequently, a second electron density 1706 is updated, the second electron density 1706, which is the post-update second electron density, is set as the fifth electron density, and the electron density estimator 108 is trained. That is, when the direction H2 goes away from the direction H1, the second electron density 1706 is updated to change the direction H2 toward the direction to the convergence value 1705, and the electron density estimator 108 can be trained. Consequently, efficient training can be achieved.

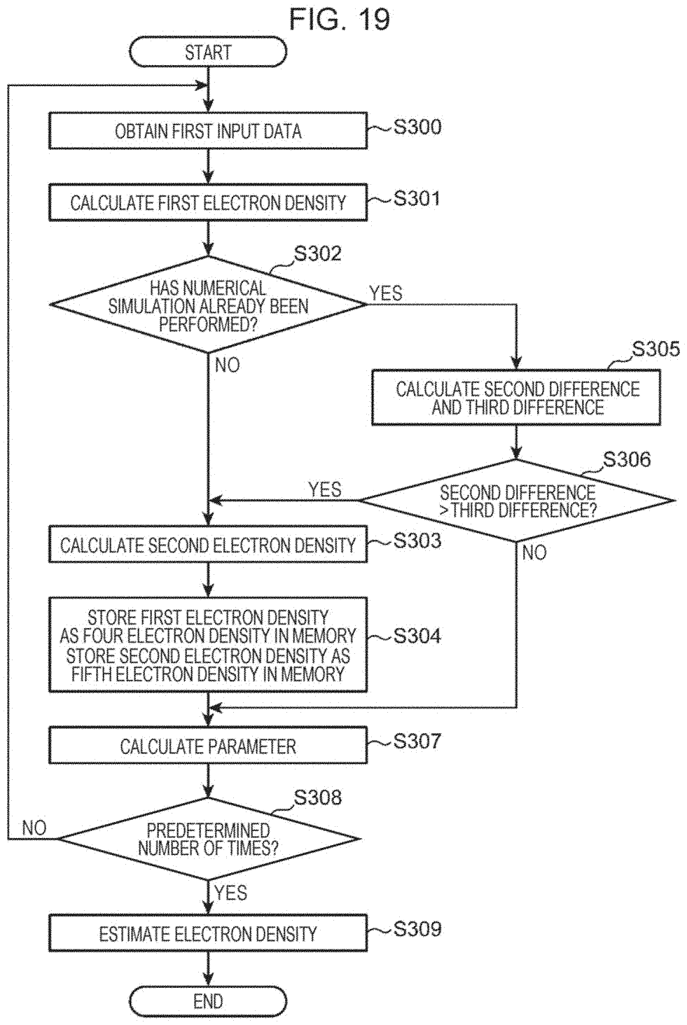

FIG. 19 is a flow chart illustrating an example of processing according to the second embodiment of the present disclosure. In the following, using FIG. 19, processing for training the electron density estimator while skipping the numerical simulation will be described.

Processing in steps S300 and S301 is the same as that in steps S100 and S101 in FIG. 15, and thus the description thereof will be omitted.

### Step S302

The electron density updater 103 determines whether the numerical simulation has already been performed on the first input data. In a case where the numerical simulation has not been performed (NO in step S302), the process proceeds to step S303. In a case where the numerical simulation has been performed (YES in step S302), the process proceeds to step S305. In this case, cases in which a determination NO is obtained in step S302 include a case where the numerical simulation according to the density functional method has not been performed at all. Such a case happens, for example, right after the procedure of FIG. 19 starts.

### Step S303

The electron density updater 103 sets the first electron density calculated in S301 as the initial electron density of the density functional method, sets, as calculation conditions for the density functional method, the first input data input to the electron density estimator 108 when the first electron density is calculated, and performs the numerical simulation according to the density functional method to calculate a second electron density. In this case, in one loop from step S100 to step S104, the electron density updater 103 performs the calculation of the numerical simulation one time. That is, the electron density update calculation in the density functional method is performed one time. Note that this is an example, and this calculation may be performed multiple times in one loop.

### Step S304

The electron density updater 103 stores, in a memory, the first electron density used when the second electron density is calculated in the last numerical simulation, that is, in processing in step S303 as a fourth electron density. In addition, the electron density updater 103 stores, in the memory, the second electron density obtained when the last numerical simulation, that is, processing in step S303 is performed as a fifth electron density.

### Step S305

The electron density updater 103 calculates a second difference, which is the difference between the first electron density and the fourth electron density. In addition, the parameter calculator 104 calculates a third difference, which is the difference between the first electron density and the fifth electron density. In this case, the first electron density indicates the latest first electron density calculated in step S301. In this case, similarly to as in the case of the first difference, for example, an RMSE can be used as the second difference and the third difference. Note that this is an example, and the second difference and the third difference may be calculated using any method as long as the method can evaluate errors such as the MAE.

### Step S306

The parameter calculator 104 determines whether the second difference is greater than the third difference. In a case where the second difference is greater than the third difference (YES in step S306), the process proceeds to step S303. In a case where the second difference is less than or equal to the third difference (NO in step S306), the process proceeds to step S307. In this case, the condition that the second difference is greater than the third difference is an example of predetermined conditions.

### Step S307

Immediately after the second electron density is updated in step S304, similarly to as in step S103, the parameter calculator 104 calculates the first difference between the second electron density (the fifth electron density) and the first electron density (the fourth electron density) used to calculate the second electron density and updates the parameter W_t for the electron density estimator 108 such that the first difference is minimized.

In contrast, in a case where it is not immediately after the second electron density is updated, the parameter calculator 104 updates the parameter W_t for the electron density estimator 108 such that the third difference calculated in step S305 is minimized.

Processing in steps S305 and S306 is the same as that in steps S104 and S105 in FIG. 15, and thus the description thereof will be omitted.

Next, the overview of the procedure in FIG. 19 will be described. First, the procedure is started, and then it is determined to be NO in step S302 since the numerical simulation has not yet been performed. In step S303, the numerical simulation is performed to calculate a second electron density. Thereafter, until the second difference becomes greater than the third difference, the loop constituted by S302 (YES), S305, S306 (NO), S307, and S308 (NO) is repeated, and the electron density estimator 108 is trained. Consequently, the first electron density approaches the second electron density calculated in the last numerical simulation, that is, a fifth electron density. When the second difference becomes greater than the third difference, it is determined to be YES in step S306, and the second electron density is updated by the numerical simulation performed in step S303. Thereafter, the loop constituted by S302 (YES), S305, S306 (NO), S307, and S308 (NO) is repeated until the second difference becomes greater than the third difference.

In this manner, in the present procedure, in a case where the second difference is smaller than the third difference, the numerical simulation is skipped, and the training of the electron density estimator 108 proceeds. At the point in time at which the second difference becomes greater than the third difference, the numerical simulation is performed to update the second electron density. Thus, in the present embodiment, the time needed to train the electron density estimator 108 can be more greatly shortened than in the case where an embodiment is used in which the numerical simulation is performed every time the first electron density is updated.

### Modification of Second Embodiment

In the second embodiment, the second electron density is updated when the second difference becomes greater than the third difference; however, the present disclosure is not limited to this. For example, the second electron density may be updated when the second difference becomes greater than the value obtained by subtracting a predetermined value from the third difference. Alternatively, for example, the second electron density may be updated when the second difference becomes greater than the value obtained by adding a predetermined value to the third difference.

The embodiments of the electron density estimation apparatus 1001 according to the present disclosure have been described above; however, the present disclosure is not limited to these embodiments. As long as not departing from the gist of the present disclosure, embodiments obtained by adding various modifications conceived by those skilled in the art to the present embodiments, embodiments formed by combining structural elements from different embodiments, and the like also fall within the scope of the present disclosure.

### Hardware Configuration

Specifically, the electron density estimation apparatus 1001 may include a computer system including a microprocessor, a read-only memory (ROM), a random access memory (RAM), a hard disk drive, a display unit, a keyboard, and a mouse. The RAM or the hard disk drive stores an electron density estimation program. The microprocessor operating in accordance with the electron density estimation program enables the electron density estimation apparatus 1001 to realize its functions. In this case, the electron density estimation program is constituted by instruction codes indicating commands to the computer in order to realize predetermined functions.

Furthermore, some or all of the structural elements of the electron density estimation apparatus 1001 may also be constituted by one system large scale integrated circuit (LSI). A system LSI is a super multi-functional LSI manufactured by integrating elements on one chip, and is specifically a computer system including, for example, a microprocessor, a ROM, and a RAM. The RAM stores computer programs. The microprocessor operating in accordance with the computer programs enables the system LSI to realize its functions.

Furthermore, some or all of the structural elements of the electron density estimation apparatus 1001 may also be constituted by an integrated circuit (IC) card or a unit module that can be attached to or detached from the computer. The IC card or the module is a computer system including, for example, a microprocessor, a ROM, and a RAM. The IC card or the module may also include the super multi-function LSI described above. The microprocessor operating in accordance with computer programs enables the IC card or the module to realize its functions. This IC card or this module may be tamper resistant.

In addition, the present disclosure may include an electron density estimation method executed by the electron density estimation apparatus 1001. In addition, this electron density estimation method may be realized by the computer executing the electron density estimation program or may also be realized by a digital signal containing the electron density estimation program.

Furthermore, the present disclosure may include a non-transitory computer-readable recording medium storing the electron density estimation program or the digital signal described above. Examples of the recording medium include a flexible disk, a hard disk, a compact disc-read-only memory (CD-ROM), a magneto-optical (MO) disk, a digital versatile disc (DVD), a DVD-ROM, a DVD-RAM, a Blu-ray Disc®, and a semiconductor memory. In addition, the electron density estimation program may contain the digital signal recorded on the non-transitory recording medium.

In addition, the present disclosure may be realized by transmitting the electron density estimation program described above or the digital signal described above through, for example, an electric telecommunication line, a wireless or wired communication line, a network, a representative example of which is the Internet, or data broadcasting.

In addition, the present disclosure may include a computer system having a microprocessor and a memory. The memory stores an electron density estimation program, and the microprocessor may operate in accordance with the electron density estimation program.

In addition, an electron density estimation program or the digital signal described above may be recorded on the non-transitory recording medium described above and be transported or may be sent via, for example, the network described above, and another independent computer system may execute the electron density estimation program or the digital signal described above.

In addition, the present electron density estimation system may include a server and a user's terminal connected to the server via a network. In this case, in FIG. 7, the electron density estimation apparatus 1001, the training DB 100, and the test DB 105 are included in the server, and the display 107 is included in the terminal. Upon obtaining, from the terminal, an electron density calculation request about a predetermined material, the electron density predictor 106 obtains second input data of the material from the test DB 105 and estimates the density of electrons of the material by inputting the second input data to the trained electron density estimator 108. It is sufficient that the server send the estimated electron density to the terminal using a communication device and display the estimated electron density on the display 107 of the terminal.

The present disclosure makes it possible to train an electron density estimator without training data and is effective in a case where a property of an unknown material is to be estimated under circumstances in which an enormous volume of training data cannot be prepared.

What is claimed is:

1. An electron density estimation method for an electron density estimation apparatus that estimates an electron density of a material from material information regarding a composition and a structure of the material,
the electron density estimation method causing a computer of the electron density estimation apparatus to execute a process, the process comprising:
(a1) obtaining first input data from a training database in which the material information is stored;
(a2) calculating a first electron density by inputting the first input data to an electron density estimator;
(a3) performing a numerical simulation using the first input data and the first electron density to calculate a second electron density, the numerical simulation being processing in which the first input data and the first electron density are set as initial values and in which electron density update processing using a density functional method is performed one or more times, the second electron density being not a convergence value obtained by the density functional method;
(a4) performing learning for the electron density estimator by calculating a parameter for the electron density estimator, the parameter minimizing a difference between the first electron density and the second electron density;
(a5) obtaining second input data from a test database in which the material information is stored, and inputting the second input data to the electron density estimator, in which the parameter is set, to estimate a third electron density; and
(a6) outputting the estimated third electron density.

2. The electron density estimation method according to claim 1,
wherein the (a5) is performed after processing from the (a1) to the (a4) is repeated a predetermined number of times.

3. The electron density estimation method according to claim 2, wherein in the (a3),
(b1) performing, in a case where the first electron density updated in the (a2) satisfies a predetermined condition, the numerical simulation to update the second electron density, and causing a process to proceed to the (a4), and
(b2) causing, in a case where the first electron density updated in the (a2) does not satisfy the predetermined condition, the process to proceed to the (a4) without updating the second electron density.

4. The electron density estimation method according to claim 3, wherein
the predetermined condition is that a second difference between the first electron density updated in the (a2) and a fourth electron density becomes greater than a third difference between the first electron density updated in the (a2) and a fifth electron density,
the fourth electron density is the first electron density used for calculating the second electron density updated in the (b1), and
the fifth electron density is the second electron density updated in the (b1).

5. The electron density estimation method according to claim 1,
wherein the material information is information described using Crystallographic Information File.

6. The electron density estimation method according to claim 1,
wherein, in the (a4), the electron density estimator is trained using the second electron density as teacher data.

7. The electron density estimation method according to claim 1,
wherein, in the (a6), an image indicating the third electron density is displayed on a display.

8. An electron density estimation apparatus that estimates an electron density of a material from material information regarding a composition and a structure of the material, the electron density estimation apparatus comprising:
a data obtainer that obtains first input data from a training database in which the material information is stored;
an electron density estimation circuit that calculates a first electron density by inputting the first input data to an electron density estimator;
an electron density updater that performs a numerical simulation using the first input data and the first electron density to calculate a second electron density, the numerical simulation being processing in which the first input data and the first electron density are set as initial values and in which electron density update processing using a density functional method is performed one or more times, the second electron density being not a convergence value obtained by the density functional method;
a parameter calculator that calculates a parameter for the electron density estimator, the parameter minimizing a difference between the first electron density and the second electron density, to perform learning for the electron density estimator;
an electron density predictor that obtains second input data from a test database in which the material information is stored and inputs the second input data to the electron density estimator, in which the parameter is set, to estimate a third electron density; and
an outputter that outputs the estimated third electron density.

9. A recording medium storing a program causing a computer to execute an electron density estimation method, the recording medium being non-transitory and computer readable, the electron density estimation method comprising:
(a1) obtaining first input data from a training database in which material information regarding a composition and a structure of a material is stored;
(a2) calculating a first electron density by inputting the first input data to an electron density estimator;
(a3) performing a numerical simulation using the first input data and the first electron density to calculate a second electron density, the numerical simulation being processing in which the first input data and the first electron density are set as initial values and in which electron density update processing using a density functional method is performed one or more times, the second electron density being not a convergence value obtained by the density functional method;
(a4) performing learning for the electron density estimator by calculating a parameter for the electron density estimator, the parameter minimizing a difference between the first electron density and the second electron density;
(a5) obtaining second input data from a test database in which the material information is stored, and inputting the second input data to the electron density estimator, in which the parameter is set, to estimate a third electron density; and
(a6) outputting the estimated third electron density.

10. An electron density estimation method performed by a computer, comprising:
(a1) obtaining first input data from a training database in which material information regarding a composition and a structure of a material and a test database are stored, the material information including the first input data, the test database including second input data;
(a2) calculating, using an electron density estimator, a first electron density D11 on a basis of the first input data and a first parameter;
(a3) performing an electron density update calculation using a density functional method one or more times to calculate a second electron density, the electron density update calculation performed one or more times including a calculation using the density functional method based on the first input data and the first electron density D11, the second electron density being not a convergence value obtained by the density functional method;
(a4) calculating a first electron density D1i on a basis of the first input data and an i-th parameter, using the electron density estimator and changing i from 2 to n;
(a5) calculating a first difference DF1j between the first electron density D1j and the second electron density, changing j from 1 to n, and detecting a smallest first difference DF1k (1≤k≤n) among the first differences DF11 to DF1n, thereby identifying the k-th parameter;
(a6) obtaining the second input data and inputting the second input data to the electron density estimator with the k-th parameter being set, to estimate a third electron density; and
(a7) outputting the estimated third electron density.
